(19) United States
(12) Patent Application Publication
(10) Pub. No.: US 2013/0325042 A1
(43) Pub. Date: Dec. 5, 2013

(54) PYLORUS PLUG AND ANASTOMOSIS

(71) Applicants: Izhak Fabian, Kfar Truman (IL); Steven Haas, Kochav Yair (IL)

(72) Inventors: Izhak Fabian, Kfar Truman (IL); Steven Haas, Kochav Yair (IL)

(21) Appl. No.: 13/484,498

(22) Filed: May 31, 2012

(51) Int. Cl. A61B 17/11

(52) U.S. Cl. 606/153

(57) ABSTRACT

A method for creating an anastomosis between a stomach and a portion of a small intestine, and simultaneously controlling passage of stomach contents through a pylorus with a pylorus plug that includes a valve operative to either close or at least partially open passageway through the pylorus.

### FIELD OF THE INVENTION

The present invention generally relates to methods for creating an anastomosis in the gastrointestinal (GI) tract, while obstructing or reducing flow of gastric contents across the pyloric valve (pylorus).

### BACKGROUND OF THE INVENTION

Magnetic anastomosis devices are used to create a channel between two viscera for the purpose of redirecting bodily fluids. For example, intestinal contents or bile may be redirected in patients who have developed an obstruction of the bowel or bile duct due to such conditions as tumor, ulcer, inflammatory strictures or trauma. Some magnetic anastomosis devices include first and second magnet assemblies comprising magnetic cores that are surrounded by thin metal rims. Due to the magnetic attraction between the two magnetic cores, the walls of two adjacent viscera (e.g., the gall bladder, common bile duct, stomach, duodenum, or jejunum) may be sandwiched and compressed between the magnet assemblies, resulting in ischemic necrosis of the walls to produce an anastomosis between the two viscera.

US Patent Application 20100292729 to Aguirre, the disclosure of which is incorporated herein by reference, describes a magnet delivery system for forming an anastomosis in the GI tract. The system includes a guidewire, a delivery catheter, an expandable balloon and a magnet. The magnet is removably secured to the delivery portion of the catheter between first and second ports.

### SUMMARY OF THE INVENTION

The present invention seeks to provide improved methods for creating an anastomosis in the GI tract, while obstructing or reducing flow of gastric contents across the pylorus, as is described more in detail hereinbelow. The method is particularly useful in a transoral gastrointestinal procedure, but can be carried out through other orifices.

There is thus provided in accordance with an embodiment of the present invention a method for creating an anastomosis between a stomach and a portion of a small intestine, and simultaneously controlling passage of stomach contents through a pylorus with a pylorus plug that comprises a valve operative to either close or at least partially open passageway through the pylorus.

### BRIEF DESCRIPTION OF THE DRAWINGS

The present invention will be understood and appreciated more fully from the following detailed description taken in conjunction with the drawings in which:

FIG. 1 is a simplified cross-sectional illustration of a prior art magnet delivery system for use in forming the anastomosis;

FIG. 2 is a simplified cross-sectional illustration of a prior art magnet delivery system for delivering two magnets, for use in forming the anastomosis;

FIG. 3 is a simplified illustration of a magnet delivery system for use in forming an anastomosis, constructed and operative in accordance with an embodiment of the present invention;

FIG. 4 is a simplified illustration of an expanded balloon of the magnet delivery system of FIG. 3, wherein the expanded balloon is used to compact walls of the jejunum to make way for a magnet;

FIG. 5 is a simplified illustration of placing another magnet in the stomach, before, during or after placement of a pylorus plug in the pylorus, in accordance with an embodiment of the present invention;

FIGS. 6 and 7 are simplified and enlarged illustrations, respectively, of magnets compressing together walls of the jejunum and the stomach to create an anastomosis; and

FIG. 8 is a simplified illustration of a pylorus plug, in accordance with an embodiment of the present invention.

### DETAILED DESCRIPTION OF EMBODIMENTS

In accordance with one embodiment of the present invention, the method includes delivery of magnets to form the anastomosis. Merely for the sake of simplicity, an exemplary embodiment is described with reference to the magnet delivery system of US Patent Application 20100292729, but the invention is not limited to this magnet delivery system.

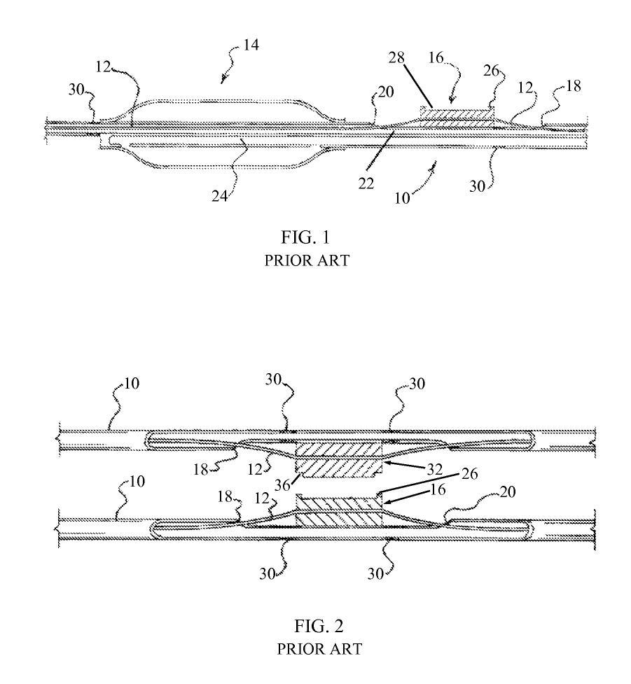

Reference is now made to FIG. 1. The magnet delivery system includes a catheter 10, a guidewire 12, and an expandable balloon 14 to deliver a magnet 16. The expandable balloon 14 is on the distal end of catheter 10 and is distal to magnet 16. Balloon 14 precedes magnet 16 during implantation so that it may dilate any strictures along the way. Once dilation has occurred, magnet 16 can be advanced to the jejunum or other desired location in the GI tract. Guidewire 12 passes through a lumen of magnet 16, first 18 and second 20 ports, and a first lumen 22 of catheter 10. Catheter 10 has a second lumen 24 that is in fluid communication with the interior of expandable balloon 14. Expandable balloon 14 can be, without limitation, non-compliant with a predetermined shape and fabricated from materials, such as but not limited to, polyethylene, polyethylene terephthalate (PET), or polyamides.

Magnet 16 is shown having a general disc shape, but other shapes, such as but not limited to, circular, cubular, cylindrical, polygonal, oval or ovoid, square and others, can also be used. Magnet 16 may include a protective coating (such as, but not limited to, polytetrafluoroethylene) for protection of the magnetic core from corrosive digestive acids or other bodily fluids.

Magnet 16 may be formed with an annular rim 26, which is slightly raised above the center of magnet 16 so as to form a basin 28 to accommodate or mate with a second magnet (as described below). In particular, when magnet 16 is delivered, rim 26 contacts the wall of the viscera and helps to initiate the ischemic necrosis of the tissue captured between magnet 16 and a mated second magnet. One or more radiopaque markers 30 may be placed on magnet 16 and/or on catheter 10 in the vicinity of magnet 16 (or other places) to mark the magnet location when viewed through fluoroscopy.

FIG. 2 illustrates two delivery systems of FIG. 1, except that in the second delivery system a second magnet 32 is affixed to another catheter 10. The second magnet 32 has an annular recess 36 that is capable of mating with the annular rim 26 of the first magnet 16.

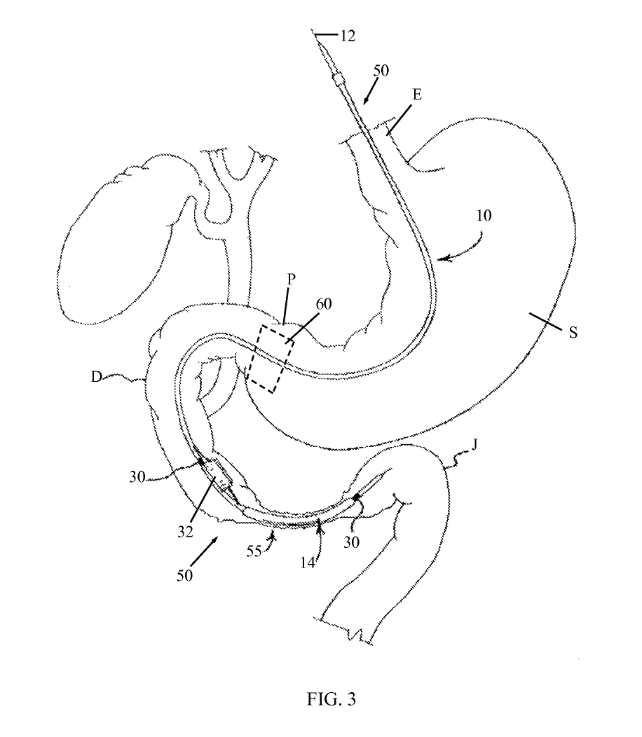

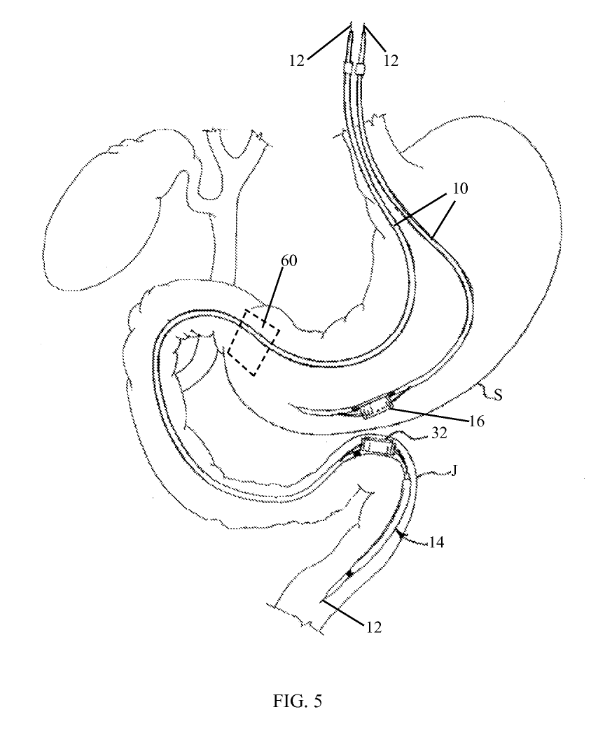

FIG. 3 illustrates a magnet delivery system 50 for use in forming an anastomosis, in accordance with an embodiment of the present invention. The exemplary embodiment is described for creating an anastomosis between the stomach and a portion of the small intestine (such as the duodenum, jejunum or ileum; the example follows for the jejunum). For simplicity, system 50 is shown for the delivery of two magnets 16 and 32 using two catheters 10 (FIG. 5), but system 50 may be used for delivering more than two magnets and at different locations. Alternatively, delivery system 50 can be used to deliver the two magnets using only one catheter.

The method for delivering the magnets to form the anastomosis includes introducing the delivery system 50 through the esophagus E, stomach S, pylorus P, duodenum D and jejunum J. In FIG. 3, delivery device 50 is shown being advanced toward a stricture 55 in the jejunum. Balloon 14 is expanded to dilate stricture 55 so that the catheter 10 can be advanced to the selected treatment site.

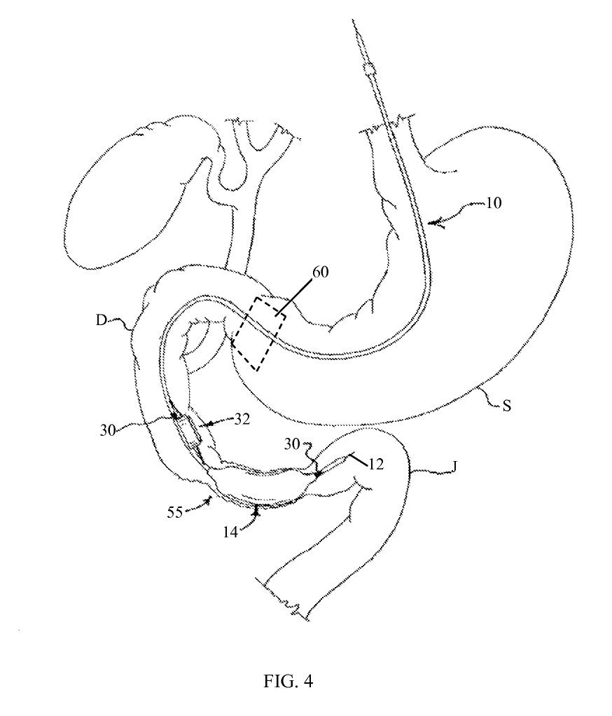

In FIG. 4, the expanded balloon 14 compacts the walls of the jejunum to make way for second magnets 32. The delivery of second magnet 32 follows once guidewire 12 has been positioned adjacent the wall of the jejunum, as seen in FIG. 5. Second magnet 32 is placed on dilation catheter 10 (FIG. 2) and held in place on catheter 10 by guidewire 12. Guidewire 12 is loaded through catheter 10, passing through second port 20 (FIG. 2) in the catheter lumen, through the lumen of second magnet 32, and then reentering the catheter lumen through first port 18. Using the radiopaque markers 30 as a guide, catheter 10 is advanced such that second magnet 32 is placed adjacent to the wall of the jejunum as shown in FIG. 5.

In accordance with an embodiment of the invention, a pylorus plug 60 is introduced and positioned in the pylorus. As is well known in the art, the pylorus defines the passageway between the stomach and the duodenum and includes a pyloric sphincter for controlling the passage of stomach contents into the intestines. As used herein, “pylorus” generally refers to the area of the opening from the stomach to the duodenum, and includes locations both upstream (proximal) of the pyloric sphincter and downstream (distal) of the pyloric sphincter.

Pylorus plug 60 may be advanced over the same guidewire 12 of catheter 10 as second magnet 32. Alternatively, pylorus plug 60 may be advanced over another guidewire (not shown). Accordingly, pylorus plug 60 may be positioned in the pylorus before, during or after placement of second magnet 32 in the jejunum.

Pylorus plug 60 is also shown in FIG. 8. In one embodiment, pylorus plug 60 includes a stent body 62, which may be without limitation, a self-expanding stent or a balloon-expandable stent. The stent body 62 is adapted to expand against the walls of the pylorus to anchor pylorus plug 60 in place. Stent body 62 may optionally have barbs or other protrusions 64 (or other devices, such as hooks, rings, roughened surfaces, etc.) for enhancing fixation. Pylorus plug 60 includes a lumen 66 which is opened or closed with a valve 68. Valve 68 may be remotely controlled by an external device (such as being electronically or magnetically controlled), or may be controlled manually by a suitable push-pull wire and the like. Pylorus plug 60 may also be provided with one or more radiopaque markers.

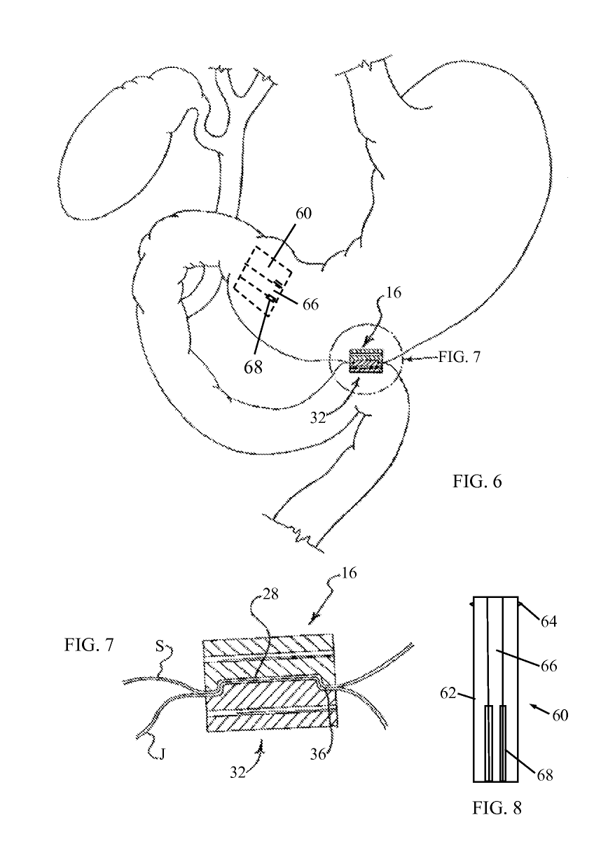

Reference is now made to FIG. 5. Before, during or after placement of pylorus plug 60 in the pylorus, the first magnet 16 is introduced into the stomach over guidewire 12, in a similar manner to introducing second magnet 32 first into the stomach. First magnet 16 is positioned adjacent the wall of the stomach that borders the jejunum near the location of second magnet 32. To release the first and second magnets 16 and 32, the operator removes guidewire 12 and then catheter 10. Magnets 16 and 32 are released so that the magnetic forces attract the magnets together, compressing together the walls of the jejunum and the stomach as seen in FIGS. 6 and 7. When implanted and mated with first magnet 16, second magnet 32 fits within rim 26 of first magnet 16.

Once the necrosis of the walls of the stomach and the jejunum is complete, an anastomosis is formed. The magnets 16 and 32 can then pass through the body naturally or can be removed by means such as laparoscopic removal, endoscopic removal, or other procedure.

The pylorus plug 60 can be left closed, i.e., valve 68 is completely closed so as to block passage of stomach contents therethrough. With pylorus plug 60 completely closed, the only route for stomach contents to flow to the jejunum is through the anastomosis. Alternatively, pylorus plug 60 can be partially or completely opened during or after completion of the anastomosis. In such a case, the stomach contents have two paths to flow through to arrive at the jejunum; valve 68 controls the amount that flows through the pylorus.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A method comprising:
creating an anastomosis between a stomach and a portion of a small intestine, and simultaneously controlling passage of stomach contents through a pylorus with a pylorus plug that comprises a valve operative to either close or at least partially open passageway through the pylorus.

2. The method according to claim 1, wherein creating the anastomosis comprises delivering a first magnet to the stomach and a second magnet to the portion of the small intestine, and causing said magnets to attract one another so as to compress together walls of the portion of the small intestine and the stomach, causing necrosis of the walls of the stomach and the portion of the small intestine and creating the anastomosis.

3. The method according to claim 2, wherein said magnets are delivered with separate catheters and guidewires.

4. The method according to claim 2, wherein said magnets are delivered with a common catheter.

5. The method according to claim 1, further comprising controlling opening and closing of said valve by an external remote control.

6. The method according to claim 2, further comprising using an expandable balloon to dilate a stricture to enable passage of at least one of said magnets therethrough.

7. The method according to claim 2, further comprising using radiopaque markers to determine a position of at least one of said magnets and said pylorus plug.
